(19) United States
(12) Patent Application Publication
(10) Pub. No.: US 2004/0086990 A1
(43) Pub. Date: May 6, 2004

(54) Method for increasing the intracellular glutamate concentration in yeast

(76) Inventors: Rogier Meulenberg, En Delft (NL); Geoffroy Hartmann, Rotterdam (NL)

Correspondence Address: MORRISON & FOERSTER LLP, 3811 VALLEY CENTRE DRIVE, SUITE 500, SAN DIEGO, CA, 92130-2332, US

(21) Appl. No.: 10/468,926

(22) PCT Filed: Feb. 22, 2002
(86) PCT No.: PCT/EP02/01982

(30) Foreign Application Priority Data
Feb. 26, 2001 (EP) 01000028.9

(51) Int. Cl. C12P 7/06; C12N 1/18

(52) U.S. Cl. 435/161; 435/254.21

(57) ABSTRACT

The present invention provides a method for the fermentation of yeast, the method comprising fermenting yeast cells in a volume of more than 10 m³; wherein: for more then half of the fermentation time the fermentation temperature is at least 30° C; and the dissolved oxygen tension (DOT) is maintained at more than 5% during at least part of the fermentation.

### FIELD OF THE INVENTION

The present invention relates to a method for increasing the intracellular concentration of glutamate in a yeast cell. The invention further relates to yeastcells obtained by this method, and yeast extracts derived from the yeast cells.

### BACKGROUND OF THE INVENTION

Monosodium glutamate (“glutamate”) is widely known as a taste and flavor enhancer. For example, exogenous glutamate has been added to yeast extrads in order to improve their taste performance. Yeast extracts derived from yeast with a high intracellular concentration of endogenous glutamate have been prepared. For example, in EP-A1-0 805 202 a process for obtaining a mutant yeast strain which accumulates glutamate is referred to. A mutant yeast strain of the genus Saccharomyces having a resistance to a glutamic acid antagonist was produced by treating the yeast with a mutagen, such as nitroguanidine, UV or X-ray irradiation and subsequently, selecting the strain in which a large amount of glutamate was accumulated. In addition, in EP 0 592 785 A2 yeast, and in particular Saccharomyces cerevisiae, is referred to, which is also obtained by mutagenesis and selection in the presence of a glutamic acid antagonist.

### SUMMARY OF THE INVENTION

The object of the present invention is to provide a simple and efficient method for increasing the intracellular glutamate concentration of yeast cells.

This is achieved by fermenting the yeast cell in a fermentation medum for at least part of the time at a fermentation temperature of at least 30° C., and a dissolved oxygen tension (DOT) of at least 5%. 100% DOT means that a medium (or liquid) is saturated with air corresponding to approximately 0.25 mM dissolved O₂ (at 0° C. and 0.1 Mpa).

Accordingly, the present invention provides a method for the fermentation of yeast, the method comprising fermenting yeast cells in a volume of more than 10 m³ wherein:

for more then half of the fermentation time the fermentation temperature is at least 30° C.; and

the dissolved oxygen tension (DOT) is maintained at more than 5% during at least part of the fermentation.

The present invention also provides a yeast obtainable by the method of the invention as well as a yeast extract derived from such a cell.

The present invention further provides a food or drink comprising, or produced using, a yeast cell or yeast extract of the invention.

### DETAILED DESCRIPTION OF THE INVENTION

According to the invention it has surprisingly been found that the intracellular concentration of glutamate in yeast cells can be increased by fermenting the yeast cells under particular selected fermentation conditions, preferably under an increased fermentation temperature in combination with an increased oxygen tension (expressed as the dissolved oxygen tension) as compared to conventional fermentation conditions.

With the method according to the invention, yeast cells with an increased intracellular glutamate concentration are produced by aerobically fermenting the yeast cells at the selected fermentation conditions in a suitable fermentation medium. In general a carbon and a nitrogen source are fed continuously into the medium. Inorganic substances, amino acids and vitamins may be added as required. The fermentation process may be for example a fed-batch fermentation which is carried out with incremental feeding of the growth substrate. Examples of the carbon source to be used in the method of the invention include glucose, fructose and molasses. Examples of a suitable nitrogen source include ammonia, ammonium salts, such as ammonium sulfate, carbonate and acetate, and urea. The fermentation is an aerobic process wherein oxygen is, for example, supplied through an oxygen-containing gas such as air, or oxygen-enriched air into the fermentation vessel. By fermentation it is meant the last step of culturing a yeast, that is the step of culturing the yeast before harvesting the yeast. The fermentation process of the invention is typically conducted in fermenters of production scale. Fermenters of production scale typically have volumes of more than 10 m³.

Typically, the fermentor will have a volume of from 10 to 1000 m³, preferably from 10 to 500 m³, more preferably from 10 to 250 m³. Such a fermentation is meant herein as fermentation on production scale.

In general there are several steps of culturing yeast starting from a solid culture, through shake flask cultures and/or minifermentor cultures which result in the production of yeast cells that are suitable for incubation in the production fermentation process. All fermentation steps for culturing the yeast prior to the last fermentation process are in general mentioned as culturing processes and any of them, or indeed all of them may be used to prepare yeast for use in the invention. The last fermentation can be performed on a production scale (production fermentation) in a fermentor of more than 10 m³ or on laboratory scale in a fermentor of less than 1000 litres. Between 1000 litres and 10 m³ is pilot (plant) scale.

Analogously fermentation medium means the medium used in the last fermentation process. As fermentation temperature is meant the temperature used in the last fermentation process. The present invention is solely linked to the conditions used in the production fermentation process resulting in a broth comprising yeast which may be used for the production of yeast extracts.

In a particularly preferred embodiment of the method according to the invention the dissolved oxygen tension (DOT) is maintained from 10 to 50%, typically from 15 to 30%, preferably from 10 to 25%. The DOT is typically at least 10%, preferably at least 15%, more preferably at least 20% during the last fermentation process on production scale. The increased DOT (5% DOT or more) is maintained for at least half of the fermentation time preferably for at least 60% and even more preferably for at least 80% of the fermentation time. The intracellular glutamate concentration may be increased by, for example from 25 to 35% in comparison to yeast cells that are fermented at a conventional fermentation temperature and dissolved oxygen tension.

According to the invention the fermentation temperature may be any fermentation temperature above 30° C. for at least half of the time of the fermentation on production scale. Preferably for at least 60%, more preferably for at least 80% of the production time. The fermentation temperature preferably is at least 33° C. and at most 38° C. At higher temperature above 38° C. cell growth will be reduced. Typically the temperature is from 31 to 38° C., preferably from 33 to 36° C. and more preferably from 34 to 36° C.

In another preferred embodiment of the method according to the invention, the temperature during the fermentation process is increased from an initial fermentation temperature to a final fermentation temperature. Preferably the initial temperature is from 28 to 33° C., more preferably from 29 to 31° C. and the final fermentation temperature is from 33 to 38° C., more preferably from 35 to 37° C. Most preferably the initial fermentation temperature is 29° C. and the final fermentation temperature is 36° C. The increased DOT (more than 5% DOT) and the increased temperature (30° to 38° C.) are preferably maintained at the same time or almost the same time. The temperature can be increased on average from 0.3 to 3° C./hour, preferably from 0.5 to 2° C./hour and most preferably 1° C./hour. The increase of temperature will be started soon after the start of the last fermentation. Preferably within two hours after the start, more preferably within one hour, even more preferably the increase starts immediately.

The yeast cell that preferably is used in the method according to the invention belongs to the genus Saccharomyces, more preferably the yeast cell is a Saccharomyces cerevisiae. The strain may be a mutant yeast strain selected for its ability to produce increased levels of glutamate in comparison to wild type strains. Alternatively, it is a wild type strain.

The invention further relates to the yeast cells, obtainable by the method of the present invention. The yeast cells can be recovered from the fermentation medium by conventional means, such as for example centrifugation or filtration. Said yeast cells can, for example, be used for the preparation of yeast extracts.

In addition, the invention relates to yeast extracts that are derived from the yeast cells according to the invention. Yeast extracts are concentrates of the soluble components of yeast cells and can for example advantageously be used in the fermentation industry as substrates, and in the food industry as flavour improvers, as flavour enhancers, or even as pure flavours. Yeast extract can for example be produced by autolysis or hydrolysis (e.g. preceded by a heatshock to kill yeast specific enzymes). The invention also provides food or drink comprising, or produced using, a yeast cell or extract of the invention.

The invention is further illustrated by the following example and figures.

FIG. 1 is a diagram showing the effect of increasing fermentation temperatures on glutamate levels, RNA and yield in Saccharomyces cerevisiae; (labscale fermentation).

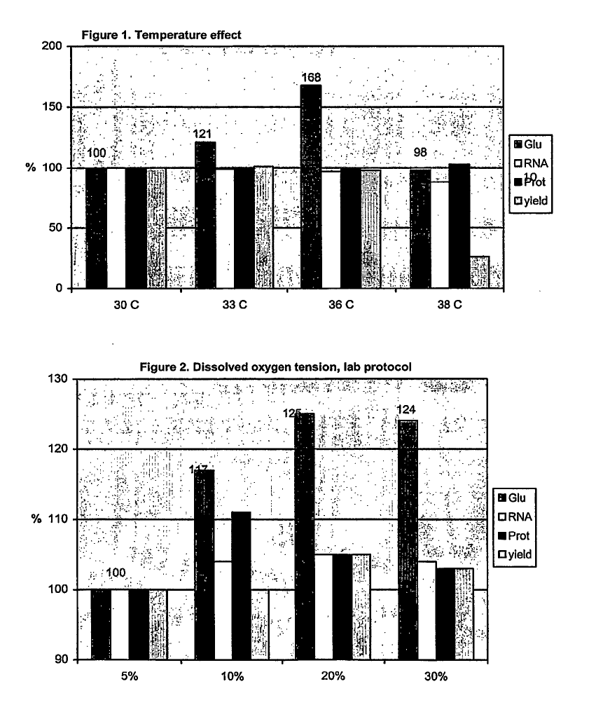

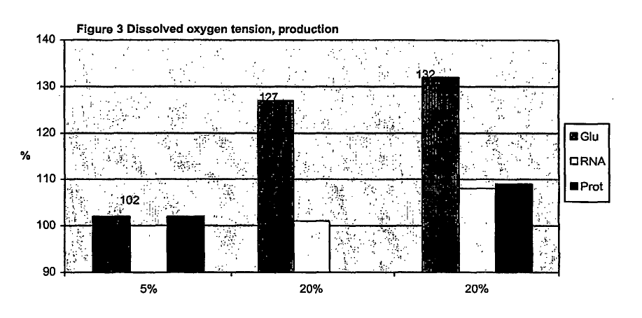

FIG. 2 shows the effect of increasing oxygen concentration (expressed as the dissolved oxygen tension (DOT)) on glutamate levels in yeast in labscale fermentations; and

FIG. 3 is a diagram showing the effect of increasing oxygen concentrations on glutamate levels in yeast in production scale fermentations.

### EXAMPLE

Fermentation Temperature

### Reference Example

Labscale fermentation experiments were performed. The labscale fermentation process was based on a direct downscale of the production scale fermentation process usually employed.

A culture of a yeast strain was grown in a series of fermentors. Cells were cultivated in 10 litres laboratory fermentors with a net culture volume of 6 litres. During the fermentation the pH was maintained at the desired values by automatic control. The fermentation recipe used was based on procedures as described by Butscheck and Kautzmann, Die Hefen, Band II Technology der Hefen, p. 501-591 (1962), Verlag Hans Carl, Nürnberg, FRG, and Reed and Peppler in Yeast Technology, the AVI Publishing Company Inc., Westport, Conn. USA (1973).

The cultivation conditions for the final fermentations were:

molasses consisting of 80% by weight of beet molasses and 20% by weight of cane molasses, calculated on the basis of 50% sugar;

the required amount of phosphate was added in the form of potassium dihydrogen phosphate, prior to inoculation;

nitrogen was supplied during the fermentation as a 10% solution of ammonia in water according to Table 1;

the pH was kept at 3.1 during the first 1.5 hours of fermentation and increased thereafter according to Table 1 to 5.2 by the end of the fermentation.

per kg of molasses containing 50% fermentable sugars, 12 mg of vitamin B1, 18 mg of vitamin B₆ and 0.24 mg biotin was added prior to inoculation.

TABLE 1
Fermentation recipe used for batchwise production of yeast

| Hours after | Molasses supply (% of | | Ammonia supply (% of |
|---|---|---|---|
| inoculation | total amount added) | pH | total amount used) |
| <0 | 0 | 3.1 | 8 |
| 0-1 | 3 | 3.1 | 7 |
| 1-2 | 3 | 3.3 | 6 |
| 2-3 | 4 | 4.4 | 7 |
| 3-4 | 5 | 4.8 | 6 |
| 4-5 | 6 | 4.8 | 7 |
| 5-6 | 7 | 4.8 | 6 |
| 6-7 | 8 | 4.8 | 6 |
| 7-8 | 9 | 5.2 | 7 |
| 8-9 | 9 | 5.2 | 7 |
| 9-10 | 9 | 5.2 | 6 |
| 10-11 | 8 | 5.2 | 7 |
| 11-12 | 9 | 5.2 | 6 |
| 12-13 | 9 | 5.2 | 7 |
| 12-14.25 | 11 | 5.2 | 7 |

In order to determine the influence of the fermentation temperature on the intracellular glutamate concentration, Saccharomyces yeast cells were fermented in labscale fermentations under different fermentation conditions: a constant growth temperature of 30° C. (control), 33° C., 36° C. or 38° C. and by implementing a temperature profile of 29-32 ° C. (+1° C./hour) or a 29-36° C. (+1° C./hour). In the temperature profiled fermentations the beginning of the fermentation, the temperature was increased. When the final temperature was reached, this temperature is maintained until the end of the fermentation.

It was demonstrated that when yeast cells were fermented at least partly at a constant fermentation temperature of 33° C. or 360° C. an increased glutamate concentration in the yeast cells could be detected compared to yeast cells that were fermented at a fermentation temperature of 30° C. RNA, protein and biomass yield on sugar remained unaffected (FIG. 1). Thus, in labscale fermentations, at a fermentation temperature of 33° C. or 36° C. an increase in intracellular glutamate content of 21% and 68%, respectively, was achieved. At a constant temperature of 38° C. ethanol production was triggered and the yield of sugar significantly decreased.

By increasing the fermentation temperature during the fermentation from an initial fermentation temperature to the final fermentation temperature, i.e. by implementing a linear temperature profile from 29° C. to 36° C. (+1° C./hr)) a 29% increase in intracellular glutamate concentration was achievable.

The data thus indicates that in labscale fermentations a fermentation temperature higher than 30° C. has a strong impact on the intracellular glutamate content of S. cerevisiae.

Based on these results, quadruplicate production scale fermentations (105 m³ fermentation) were performed with an increased temperature profile of 29° C. -36° C.

The final fermentations were carried out under aerobic conditions and with incremental feeding of molasses and nitrogen. Yeast cells were fermented in fed batch fermentations which were characterized by carbon limitation. The limitation was controlled by an exponentially increasing molasses feed (constant growth rate) until the oxygen uptake rate limited the productivity. The molasses feed was then held constant until the end of the fermentation process. Nitrogen is provided by a constant ammonia feed during the fermentation. During the fermentation process the molasses and ammonia feed were decreased when ethanol concentration exceeds 0.02%.

All quadruplicate fermentations were technically successful, i.e. all factors fell within the standard deviation, no output loss occurred. However, the intracellular glutamate content in the yeast cells was not increased. There was no repetition of the increasing in intracellular glutamate seen in the lab scale sized fermentations, In table 2, average yeast characteristics for the fermentations are summarised.

TABLE 2
Yeast characteristics

| | Glutamate | RNA | Protein |
|---|---|---|---|
| Experiment | [% on DW] | [% on DW] | [% on DW] |
| A | 1.8 | 8.3 | 62 |
| B | 3.0 | 8.4 | 62 |
| C | 1.7 | 8.2 | 63 |

A: Fermentation temperature 29-32° C. (+1° C./hr increase), DOT 0-5%, production scale (100 m³)
B: Fermentation temperature 36° C., DOT 20%, laboratory scale
C: Fermentation temperature 29-36° C. (+1°/hr increase), DOT 0-5%, production scale (100 m³)

Although an increased fermentation temperature at labscale fermentation conditions led to an increase in intracellular glutamate concentration in yeast, this effect was not observed after scaling up the fermentation to a production fermentation process. In pilot plant scale productions the same increase in intracellular glutamate was observed as in labscale.

Dissolved Oxygen Tension on Intracellular Glutamate

The main difference between labscale fermentation and production fermentations is the dissolved oxygen tension (DOT employed). The fermentations at labscale are characterized by a DOT regulation at 20% air saturation, whereas the DOT level during a production fermentation process generally fluctuates between 0 and 5% after 6 hours until the end of the fermentation.

In order to investigate the influence of DOT on intracellular glutamate levels labscale experiments were performed at a fermentation temperature of 29° C.-36° C. and with increasing oxygen concentration (DOT of 5, 10, 20 and 30% respectively). It was thus demonstrated that the dissolved oxygen concentration during the fermentation can have a high impact on the glutamate content in yeast (FIG. 2).

### Example

The effect of the oxygen concentration on the glutamate levels under production scale conditions (100 m³), using feed and inoculation adjustment to increase the DOT level to 20% saturation, at a fermentation temperature of 29-36° C. (+1° C./hr). An increased DOT was achieved with a 20% decreased molasses and ammonia feed. In order to keep a similar grown pattern, the number of yeast cells in the inoculum was also decreased by 20%. Under these fermentation conditions, a fermentation product and final extract were obtained containing approximately 25%-35% higher levels of glutamate than those achieved in the 5% DOT fermentation (FIG. 3). The yield of biomass on sugar remained similar.

Surprisingly, yeast cells that were fermented in production fermentations according to the method of the present invention gave a fermentation product and final extract containing approximately 25-35% higher levels of glutamic acid as shown in FIG. 3. These results are confirmed by yeast extract preparation leading to an increase in glutamate concentration from 3.0% (based on dry weight) to 3.9% (based on dry weight).

According to the invention yeast cells with an increased glutamate content can be obtained by employing the fermentation conditions of the invention, in particular by fermenting the yeast cells at least partly at an increased fermentation temperature of more than 30° C., in combination with maintaining the dissolved oxygen tension during at least part of the fermentation process at higher than 5%.

1. A method for the fermentation of yeast, the method comprising fermenting yeast cells in a volume of more than 10 m³ wherein:
for more then half of the fermentation time the fermentation temperature is at least 30° C.; and
the dissolved oxygen tension (DOT) is maintained at more than 5% during at least part of the fermentation.

2. A method according to claim 1, wherein the dissolved oxygen tension is maintained at at least 20% for at least part of the fermentation.

3. A method according to claim 1 or 2, wherein the fermentation temperature is from 32° C. to 38° C.

4. A method according to any one of the preceding claims wherein the fermentation temperature is 36° C.

5. A method according to any one of the preceding claims, wherein during the fermentation process the temperature is increased from an initial fermentation temperature to a final fermentation temperature.

6. A method according to claim 5, wherein the initial fermentation temperature is from 28° C. to 33° C. and the final fermentation temperature is from 33° C. to 38° C.

7. A method according to claim 5 or 6, wherein the initial fermentation temperature is 29° C. and the final fermentation temperature is 36° C. and the temperature is increased by 1° C. per hour in the transition from the initial to the final temperature.

8. A method according to any of the preceding claims, wherein the yeast cell belongs to the genus Saccharomyces.

9. A method according to claim 8, wherein the yeast cell is Saccharomyces cerevisiae.

10. A method according to claim 8 or 9, wherein the yeast strain does not comprise a mutation in a gene which is involved in, or influences, intracellular glutamate concentration.

11. A yeast cell, obtainable by a method according to any of the preceding claims.

12. A yeast extract derived from a yeast cell according to claim 11

13. A food or drink comprising, or produced using, a yeast cell according to claim 11 or a yeast extract according to claim 12.
